(19) United States
(12) Patent Application Publication
(10) Pub. No.: US 2007/0160719 A1
(43) Pub. Date: Jul. 12, 2007

(54) Method of determining the source of bacteria

(76) Inventor: Darrell L. Bigalke, Stillwater, MN (US)

Correspondence Address: MERCHANT & GOULD PC, P.O. BOX 2903, MINNEAPOLIS, MN, 55402-0903, US

(21) Appl. No.: 11/520,279

(22) Filed: Sep. 12, 2006

Related U.S. Application Data
(60) Provisional application No. 60/759,221, filed on Jan. 12, 2006.

(51) Int. Cl. G01N 33/02

(52) U.S. Cl. 426/231; 426/522

(57) ABSTRACT

A method of determining the source of spore-forming bacteria. The method including obtaining a raw milk sample in a oxygen-permeable bag, and associating the milk bag sample (i.e., raw milk sample and oxygen-permeable bag) with a particular raw milk source. The method further including conducting a test to determine the existence of unacceptable levels of spore-forming bacteria in the source-associated milk bag sample so that steps can be taken to control repeated contamination.

### CROSS-REFERENCE TO RELATED APPLICATIONS

This application claims the benefit of U.S. Provisional Application Ser. No. 60/759,221, filed Jan. 12, 2006; which application is incorporated herein by reference.

### FIELD OF THE INVENTION

This disclosure relates to methods and devices for use in the dairy industry. More specifically, this disclosure relates to methods and devices for use in detecting spore-forming bacteria in milk products.

### BACKGROUND OF THE INVENTION

The food industry utilizes a number of methods and apparatuses for obtaining and testing food product samples to assure the quality of food products. In the dairy industry, to assure the quality of milk products, product samples are often monitored for unacceptable levels of microbial contamination and spoilage microorganisms.

In conventional methods of monitoring for milk product quality, milk product samples are taken at the dairy plants from large vessels or containers. The milk product samples can be in the form of a raw ingredient sample, or in the form of a resulting end product sample. Tests are conducted on the milk product sample to determine whether the product is acceptable for sale and consumption. If the test indicates that the milk product sample is unacceptable, or contaminated, the entire volume of milk product is rendered unusable. Vessels often hold a significant volume of product amassed from a number of sources. Contamination of the entire volume of product can often be caused by only a single source, resulting in significant waste of the amassed milk product.

Improvement in methods and devices for sampling is needed, generally to better accommodate: contamination source identification, and management and control of product contamination.

### SUMMARY OF THE INVENTION

The principles described herein relate to efficient and effective techniques and apparatus for obtaining aseptic samples for the purpose of monitoring the quality of various products. In particular, this disclosure relates to methods and devices for aseptically monitoring contamination of a liquid by bacteria, which grow in the presence of oxygen. The aseptic samples obtained by the presently disclosed method are typically in the form of liquid, while the finished goods may be in the form of liquid, solid, or gas.

Such methods and devices can be applied in industries such as, but not limited to, the pharmaceutical, bioengineering/biotechnology, brewing/distilling, cosmetic and personal care, food processing and dairy processing industries. The industries to which this disclosure can apply may be associated with products that are ingested by, or applied topically to, humans or animal consumers; and can also be associated with non-ingestible and non-topically applicable products, where such product contamination is harmful or undesirable (e.g., odoriferous products, paints, and other consumer products).

One aspect of the present invention relates to a method of determining the source of spore-forming bacteria. The method includes the steps of obtaining a raw milk sample within a oxygen-permeable bag from a particular source, associating the oxygen-permeable bag sample with the particular source, and monitoring the quality of raw milk from the particular source to control the quality of a food product at a processing plant.

A variety of examples of desirable product features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE DRAWINGS

FIG. 1 is a block diagram of a method for determining the source of spore-forming bacteria in milk, in accordance with the principles disclosed.

### DETAILED DESCRIPTION

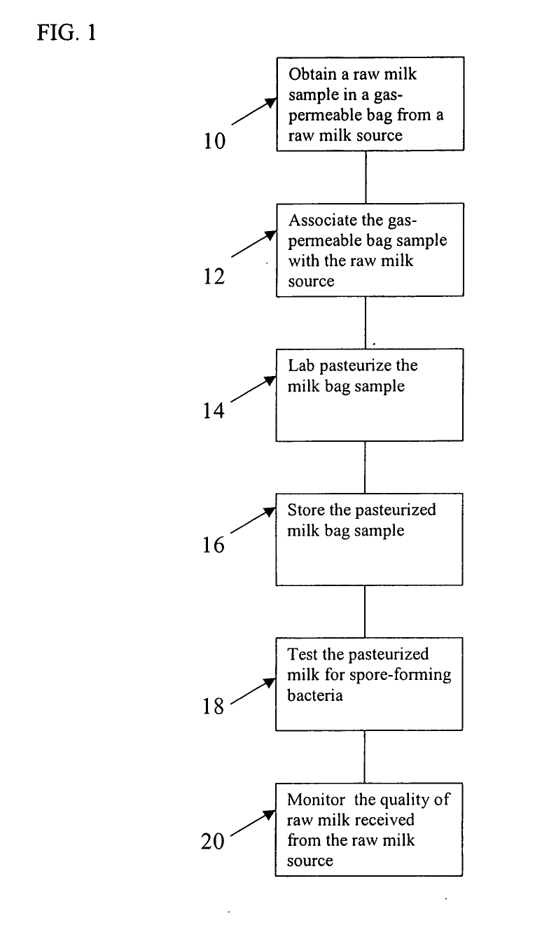

Referring to FIG. 1, the present disclosure relates to a method of determining the source of heat resistant bacteria, such as spore-forming bacteria, which can contaminate a food product and the equipment used in the production of the food product. While the preferred method will be described in application to, and use in, the dairy industry, it is to be understood that the method is not to be construed as limited to use in the dairy industry.

Heat resistance bacteria, such as spore-forming bacteria is inherent in raw milk; however, an excessive amount of spore-forming bacteria, or the presence of spore-forming bacteria that has an accelerated growth rate is undesirable. In particular, an excessive amount of gram-positive psychrotrohic bacteria, which causes spore-forming bacteria, or gram-positive psychrotrohic bacteria that has an accelerated growth rate is undesirable.

Spore-forming bacteria is generally caused by contamination introduced into pre-pasteurization milk processes and cold milk handling equipment. Cold milk handling equipment includes milk silos, bulk tanks, and tanker trucks that store or contain pre-pasteurization milk at cold temperatures. Gram-positive psychrotrohic bacteria that causes spore-forming bacteria can tolerate the cold temperatures of the cold milk handling equipment. The present method concerns testing for such cold-resistant spore-forming bacteria, such as gram-positive psychrotrohic bacteria.

Spore-forming bacteria in excessive amounts or having an accelerated growth rate often results in unacceptable milk quality at refrigeration temperatures. Accordingly, to improve the overall quality of milk products, it would be advantageous for the dairy industry to better control and manage spore-forming bacteria (i.e., gram-positive psychrotrohic bacteria). The disclosed method can be used in the dairy industry to control the occurrence of spore-forming bacteria contamination. Because spore-forming bacteria is inherent in raw milk, raw milk quality can greatly influence milk product quality. Accordingly, it is beneficial to monitor the level of gram-positive psychrotrohic bacteria (hereinafter referred to as spore-forming bacteria) in raw milk.

As previously described, spore-forming bacteria that affects the quality of milk products is typically caused by contamination introduced into pre-pasteurization (raw) milk processes at the dairy plants. Dairy plants often utilize large vessels that hold significant volumes of raw milk. The volume of raw milk within each of the vessels is often amassed from a number of sources. Contamination of the entire volume of raw milk of a vessel can be caused by only a single source, resulting in significant waste of the amassed raw milk or raw milk product. Controlling the occurrence of spore-forming bacteria contamination at the dairy plants by the disclosed method reduces the waste of amassed raw milk.

In the present method, as shown in FIG. 1, a sample of raw milk is obtained in a oxygen-permeable bag 10. Preferably, the raw milk is aseptically obtained. Aseptic sampling is used in applications having processes or systems that are sensitive to contamination from the outside environment, and involves the aseptic transfer of fluids between two containers or structures. Some aseptic sampling arrangements and aseptic sampling methods that can be used are disclosed U.S. Publication Nos. 2003/0110870 and 2004/0228963, which applications are incorporated herein by reference. Because the raw milk sample is aseptically obtained, contamination of both the sample and the primary volume of fluid is prevented to preclude the process of obtaining the sample as a source of spore-forming bacteria contamination. In conventional methods, glass containers are simply dipped within the large vessels so as not to preclude the process of obtaining the sample as the source of spore-forming bacteria contamination.

In the present method, the sample of raw milk is obtained by at least partially filling the oxygen-permeable bag with a volume of raw milk. In one method, the amount or volume of raw milk obtained is about 2 liters. Using a volume of about 2 liters provides a sample size workable in detecting gram-positive psychrotrohic bacteria. An excessive amount of gram-positive phychrotrohic bacteria that can cause milk quality problems can be an amount as little as one bacterium per gallon of raw milk. Accordingly, utilizing a sample size of about 2 liters improves the reliability and accuracy in determining the source of the spore-forming bacteria, although other volume amounts of raw milk for purposes of sampling can be obtained in accordance with the principles disclosed. In one alternative method, the volume of raw milk obtained is about 2 liters, however, the volume is obtained in four individual samples of 250 ml each.

The oxygen-permeable bag that contains the raw milk sample may include a pouch, bag, reservoir, or other closed container. In some embodiments, the oxygen-permeable bag is correspondingly sized to the volume of raw milk obtained; in other embodiments, the oxygen-permeable bag has a large volume capacity such that the raw milk obtained only partially fills the oxygen-permeable bag. A variety of sizes and constructions of oxygen-permeable bags is contemplated.

The oxygen-permeable bag and the volume of raw milk contained within the oxygen-permeable bag define a milk bag sample. Preferably, the oxygen-permeable bag has an oxygen permeability that simulates the formation of spore-forming bacteria in the milk bag sample that would otherwise occur in the resulting milk product at a shelved refrigeration temperature. In other words, the oxygen-permeable bag preferably has an oxygen permeability that provides a level of oxygen saturation or aerobic exposure that accelerates the growth rate of spore-forming bacteria to provide an indication of the amount of spore-forming bacteria that would otherwise develop during the shelf life of the resulting milk product.

In the disclosed method, the volume of raw milk is a sample amount obtained from a particular raw milk source, as opposed to a large vessel of raw milk at a dairy plant amassed from a number of raw milk sources. In particular, the volume of raw milk can be obtained from a tanker truck, a milk silo, or a bulk storage tank, for example. As can be understood, milk silos and bulk storage tanks are associated with particular dairy farms, and are either located specifically at a particular dairy farm or used by the particular dairy farm. In the dairy industry, likewise, tanker trucks are typically associated with a particular dairy farm, or only a few dairy farms, as such trucks generally maintain a specific transportation route. Accordingly, each of the milk bag samples obtained is associated with a particular raw milk source, such as a single dairy farm or a single tanker truck that transports raw milk from only a few dairy farms.

As shown in FIG. 1, the present method includes associating the milk bag sample (i.e. the volume of raw milk contained with the oxygen-permeable bag) with a particular raw milk source 12. That is, the milk bag samples are provides an identification that associates the sample with a particular dairy farm or tanker trunk. The identification of the milk bag sample that associates the sample with the particular raw milk source can be in the form of demarcation, labeling, or bar-coding, for example. Other indicia or associating methods can be used in accordance with the principles disclosed.

Once the milk bag sample has been obtained 10, and accordingly associated with the particular milk source 12, the milk bag sample is then lab pasteurized 14. The term milk bag sample is intended to include the oxygen-permeable bag containing the amount of milk, whether pre-pasteurized (raw) or post-pasteurized. The lab pasteurizing process includes pasteurizing the milk bag sample at about 72 degrees Celcius (162 degrees Fahrenheit); typically, for a period of about 20 minutes.

Once the milk bag sample is pasteurized, the sample is stored for a time period sufficient to allow the spore-forming bacteria to grow 16 (FIG. 1). Preferably, the pasteurized milk bag sample is stored at a particular storage temperature that simulates the standard storage conditions of the milk product. For example, in one method, the milk bag sample is stored for about 10 days at an approximate temperature of about 7 degrees Celsius (45 degrees Fahrenheit). During this period, oxygen permeates the oxygen-permeable bag to oxygenate the milk sample. Oxygenating the milk sample accelerates the growth rate of any spore-forming bacteria present in the milk sample. The growth rate of the spore-forming bacteria in the milk sample is then used as an indicator of the lasting quality of the resulting milk product.

Oxygenating or exposing the milk sample to oxygen by permeation through the oxygen-permeable bag significantly accelerates the formation of spore-forming bacteria in comparison to the conventional methods of bacteria testing. In conventional methods, glass containers are used in the testing for spore-forming bacteria. Glass containers are non-permeable and require a long incubation or storage period to obtain adequate contamination results. By using the oxygen-permeable bag of the disclosed method, the time required to develop an indicating level of bacterial growth is significantly reduced due to the oxygen permeability feature of the bag. Reducing the time needed to develop bacterial growth saves in production costs associated with continuing the processing of a milk product that will subsequently be found unusable, for example. Reducing the time needed to develop bacterial growth also reduces product waste associated with combining a contaminated volume of milk with a non-contaminated volume of milk during production of a milk product, for example.

After the milk bag sample has been stored for the sufficient period of time 16, the amount of spore-forming bacteria in the volume of milk is determined 18. For example, a conventional laboratory procedure, such as a Standard Plate Count, is conducted after the period of time to determine the amount of spore-forming bacteria present within the milk sample. Methods other than the Standard Plate count for detecting spore-forming or spoilage bacteria can also be used. A level of spore-forming bacteria greater than 10,000,00 counts/ml, for instance, would indicate that the spore-forming bacteria present in the milk product has the potential for causing product quality defects.

The method of the present disclosure relates not only to determining the presence of spore-forming bacteria, but further improves upon determining the presence by determining also the source of spore-forming bacteria. In conventional methods, the results of the test are simply used to determine whether or not a quantity of milk product can be put out for sale. Conventional methods only address present or immediate quality defects. In the disclosed method, the results of testing are used to control and reduce the likelihood of future quality problems.

For example, in conventional methods, the presence of spore-forming bacteria is monitored to provide information for evaluating associated dairy plant production and cleaning processes, and a milk products shelf life. The present method further improves upon the conventional methods of monitoring spore-forming bacteria by providing or determining the source of spore-forming bacteria. By determining the source of spore-forming bacteria, the dairy plants can better control contamination by reducing the likelihood of subsequent, repeated contamination.

That is, the present method includes obtaining raw milk samples associated with particular dairy farms, for example, and determining whether a particular dairy farm is a source of spore-forming bacteria. If the test results indicate that the particular dairy farm is in fact a source of spore-forming bacteria, subsequent product received from that particular dairy farm can then be more closely monitored and tested 20 (FIG. 1). This also offers the dairy plant an opportunity to assist the particular dairy farm in resolving the farm's contamination problem.

In conventional methods, the source of contamination of the amassed volume of raw milk is not known. Therefore, the dairy plants cannot more closely monitor raw milk coming from a particular contamination source or assist a particular dairy farm in resolving the contamination problem. And, because of the delay in receiving test results due to the use of non-permeable glass containers, not only is the contaminated milk typically already fully processed, it is also likely that subsequent volumes of non-contaminated milk product have been processed through contaminated equipment. In the present method, because of the accelerated formation of bacteria and the expedited receipt of test results, the dairy plant can more quickly remedy contamination. And, unlike in conventional methods, the dairy plant is informed of the source of contamination so that steps can be taken to prevent repeated, subsequent contamination.

The above specification provides a complete description of the present invention. Since many embodiments of the invention can be made without departing from the spirit and scope of the invention, certain aspects of the invention reside in the claims hereinafter appended.

I claim:

1. A method of determining the source of spore-forming bacteria, the method comprising the steps of:
a) providing a oxygen-permeable bag;
b) obtaining a milk bag sample by at least partially filling the oxygen-permeable bag with an amount of raw milk;
c) associating the milk bag sample with a particular raw milk source;
d) pasteurizing the milk bag sample;
e) storing the milk bag sample for a period of time; and
f) determining the amount of spore-forming bacteria in the milk bag sample associated with the particular milk source.

2. The method of claim 1, wherein the step of associating the milk bag sample includes providing the oxygen-permeable bag with an identification associated with the particular raw milk source.

3. The method of claim 2, wherein the step of providing the oxygen-permeable bag with the identification includes providing the oxygen-permeable bag with an identification associated with a particular tanker truck.

4. The method of claim 2, wherein the step of providing the oxygen-permeable bag with the identification includes providing the oxygen-permeable bag with an identification associated with a particular dairy farm.

5. The method of claim 2, wherein the step of providing the oxygen-permeable bag with the identification includes providing the oxygen-permeable bag with a demarcation.

6. The method of claim 1, wherein the step of pasteurizing the milk bag sample includes pasteurizing the milk bag sample at a temperature of about 72 degrees Celsius.

7. The method of claim 1, wherein the step of storing the milk bag sample includes storing the milk bag sample for a period of about 10 days.

8. The method of claim 7, wherein the step of storing the milk bag sample includes storing the milk bag sample at about 7 degree Celsius for the period of about 10 days.

9. The method of claim 1, wherein the step of storing the milk bag sample includes storing the milk bag sample at an approximate temperature for the period of time.

10. The method of claim 1, wherein the step of storing the milk bag sample includes storing the milk bag sample for the period of time such that oxygen permeates the oxygen-permeable bag and allows formation of spore-forming bacteria present in the milk bag sample.

11. The method of claim 1, wherein the step of storing the milk bag sample further includes exposing the amount of milk of the milk bag sample to oxygen by permeation through the oxygen-permeable bag for the period of time.

12. The method of claim 1, wherein the step of obtaining the milk bag sample includes obtaining an aseptic milk bag sample.

13. The method of claim 1, wherein the particular milk source is a tanker truck, the amount of raw milk being a sample taken from the tanker truck.

14. The method of claim 1, wherein the particular milk source is a farm, the amount of raw milk being taken from a tanker truck associated with the farm.

15. The method of claim 1, wherein the particular milk source is a bulk tank, the amount of raw milk being a sample taken from the bulk tank.

16. The method of claim 1, wherein the particular milk source is a raw milk silo, the amount of raw milk being a sample taken from the silo.

17. The method of claim 1, wherein the step of at least partially filling the oxygen-permeable bag with an amount of raw milk includes at least partially filling the oxygen-permeable bag with about 2 liters of raw milk.

18. The method of claim 1, wherein the step of at least partially filling the oxygen-permeable bag with an amount of raw milk includes at least partially filling the oxygen-permeable bag with about 250 ml of raw milk.

19. The method of claim 18, further including at least partially filling additional oxygen-permeable bags with about 250 ml of raw milk.

20. The method of claim 1, wherein the step of determining the amount of spore-forming bacteria in the milk bag sample includes conducting a plate count test.
